(19) United States
(12) Patent Application Publication
(10) Pub. No.: US 2026/0219183 A1
(43) Pub. Date: Jul. 30, 2026

(54) DETECTION AND MONITORING OF SCALE FORMATION IN FLUID MIXTURES

(71) Applicant: KING FAHD UNIVERSITY OF PETROLEUM AND MINERALS, Dhahran (SA)

(72) Inventors: Ose Dimas BUDIMAN, Dhahran (SA); Shabeeb N. ALAJMEI, Dhahran (SA); Mobeen MURTAZA, Dhahran (SA); Muhammad Shahzad KAMAL, Dhahran (SA); Murtada AL-JAWAD, Dhahran (SA); Mohamed MAHMOUD, Dhahran (SA)

(73) Assignee: KING FAHD UNIVERSITY OF PETROLEUM AND MINERALS, Dhahran (SA)

(21) Appl. No.: 19/279,797

(22) Filed: Jul. 24, 2025

Related U.S. Application Data
(60) Provisional application No. 63/750,080, filed on Jan. 27, 2025.

(51) Int. Cl. G01N 21/53; G01N 21/85; G01N 21/88; G01N 33/18

(52) U.S. Cl. CPC G01N 21/534; G01N 21/8507; G01N 21/8851; G01N 33/1853; G01N 2021/855; G01N 2021/8883

(57) ABSTRACT

A method for detecting and monitoring scale formation in a fluid mixture involves combining two aqueous solutions with different ionic compositions, one rich in cations (e.g., Ca²⁺) and the other rich in anions (e.g., SO₄²⁻). Formation water (FW) with high calcium levels is mixed with seawater (SW) containing elevated sulfate concentrations. Different mixture ratios are then exposed to controlled environmental conditions that promote mineral precipitation. Light transmission is measured along the height of the fluid column over time using turbidity scanning. A localized decrease in transmission indicates scale formation. The process is assessed using the Transmission Stability Index (Tr-SI), which allows for real-time detection and analysis of scale nucleation and growth.

### CROSS REFERENCE TO RELATED APPLICATIONS

The present disclosure claims the benefit of U.S. Provisional Application No. 63/750,080, filed on Jan. 27, 2025, which is incorporated herein by reference in its entirety.

### BACKGROUND

### Technical Field

The present disclosure is directed towards scale formation monitoring in fluid mixtures, and more specifically, relates to a method for detecting and monitoring scale formation in fluid mixtures using turbidity scanning for real-time, non-invasive detection in for example oilfield operations.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Oil field scale formation poses a significant challenge for the petroleum industry, particularly during oil recovery processes. Scale formation, primarily composed of mineral precipitates such as calcium sulfate (CaSO₄), calcium carbonate (CaCO₃), barium sulfate (BaSO₄), and others, leads to numerous operational issues. The operational issues include blockages in pipelines and reservoir pores, reduced permeability, and compromised integrity of production equipment. The operational issues are further compounded in enhanced oil recovery operations where sea water is injected into underground formations to maintain reservoir pressure. The aforementioned interaction between sea water and formation water, which have different ionic compositions, accelerates scale formation. Primary drivers of scale formation include changes in temperature, pressure, and water chemistry. Such factors often lead to the supersaturation of minerals, which then precipitate, forming scale deposits. The injection of sea water introduces ions such as sulfate, which may react with calcium and strontium ions in formation water to form scale minerals such as barite and celestite. In addition, other scales such as calcium carbonate and calcium sulfate may form due to shifts in temperature and pressure during production.

Presently, various laboratory methods are employed to study and predict scale formation including static bottle tests, dynamic flow systems, and core flooding experiments, all aimed at simulating field conditions. Analytical techniques such as X-ray diffraction (XRD), scanning electron microscopy (SEM), and energy-dispersive X-ray spectroscopy (EDX) are used to identify and characterize scale minerals. While these techniques provide detailed information on the composition and morphology of scales, they do not offer real-time data on the dynamic process of scale formation in situ.

Turbidity refers to the cloudiness or haziness of a fluid caused by suspended particles, which can be an indicator of the presence of scale-forming minerals in the fluid. Turbidity scanning was previously applied to study emulsion stabilization [Rehosek, M., Laupheimer, M., and Marlow, F. Demixing behavior of Pickering emulsions stabilized by Janus particles and uniform Pickering particles at different pH values. Colloid and Polymer Science, 2023, 302 (2), 253-260]. Turbidity scanning offers real-time, non-invasive measurements of fluid clarity, making it potentially an ideal tool for early detection of scale formation in oil field operations. However, the use of turbidity scanners for scale monitoring in the context of sea water and formation water mixing has not been fully explored, leaving a gap in the field. Despite the advantages of turbidity scanning, conventional methods for detecting and managing scale formation still face significant limitations. Traditional techniques often fail to provide real-time insights, making it difficult to monitor the early stages of scale formation before blockages or operational disruptions occur. There is a clear requirement for a more immediate and accurate method that may monitor the onset and progression of scale formation in real time, enabling timely interventions to prevent further complications. This gap in the available solutions highlights the requirement for a method that not only monitors scale formation in real time but also quantifies the dynamics of mineral precipitation.

US20230280266A1 discloses a system for early detection of scaling during processing of liquid solutions. A turbidity monitor designed to measure inline turbidity is used for the solution tests as well as to detect the polymerization of silica colloids. A lamp on one side of the monitor body emits light which passes through the solution and into a detector on the other side of the monitor. The method includes measuring turbidity of a stream using the optical turbidity monitor to determine a turbidity value of the concentrate stream; calculating a first derivative of the turbidity value with respect to time to generate a turbidity derivative, determining a setpoint that corresponds to a concentration of a sealant mineral in the feed stream below which scaling of the separating element by the sealant mineral does not occur; and comparing the turbidity derivative to the setpoint. However, it does not address real-time monitoring of scale formation in fluid mixtures and the interaction between sea water and formation water in oilfield operations for early detection of mineral precipitation.

U.S. Pat. No. 10,138,715B2 discloses a well-bore monitoring process involving i) logging the temperature of a mixture of soil, rock, and brine present in a carbonate formation continuously, ii) simultaneously logging the resistivity of the mixture in the carbonate formation continuously, and iii) interpreting the logged temperature and the logged resistivity to identify dissolution of rock into the mixture and precipitation of scale from the mixture by concurrent events in the logged temperature and the logged resistivity. However, it does not address real-time monitoring of scale formation using turbidity scanning or transmission stability indices (TSI) to track mineral precipitation dynamics.

Each of the aforementioned references suffers from one or more drawbacks hindering their adoption. Accordingly, it is one object of the present disclosure to provide a method for detecting and monitoring scale formation in a fluid mixture, which overcomes the aforementioned drawbacks.

### SUMMARY

In an exemplary embodiment, a method for detecting and monitoring scale formation in a fluid mixture is described. The method includes mixing a first solution and a second solution to form the fluid mixture, where the first solution has a first Ca²⁺ concentration that is 10% or less of a second Ca²⁺ concentration of the second solution, and a first SO₄²⁻ concentration that is 1000% or more of a second SO₄²⁻ concentration of the second solution. The method further includes scanning along a height direction of the fluid mixture to measure light transmission values of the fluid mixture as a function of time and height position, and determining whether scale formation occurs in the fluid mixture based on the light transmission values.

In some embodiments, the method further includes calculating a transmission stability index [TSI or SI(tₙ)] value based on a difference between consecutive transmission profiles of the light transmission values along the height direction of the fluid mixture.

In some embodiments,

$$SI ( {t}_{n} ) = {\sum }_{j = 1}^{n} \frac{{\sum }_{{z}_{min}}^{{z}_{max}} \vert I ( {t}_{j} , {z}_{i} ) - I ( {t}_{j - 1} , {z}_{i} ) \vert}{{z}_{n}} ,$$

where SI(tₙ) is the TSI value at time of tₙ, I(tⱼ, zᵢ) is transmitted light intensity at time of tⱼ and position of zᵢ, I(tⱼ₋₁, zᵢ) is transmitted light intensity at time of tⱼ₋₁ and position of zᵢ, zₘₐₓ is a highest height position along the height direction of the fluid mixture, zₘᵢₙ is a lowest height position along the height direction of the fluid mixture, zₙ=zₘₐₓ−zₘᵢₙ and represents a height of the fluid mixture, tₙ, tⱼ and tⱼ₋₁ respectively represent time during nth, jth and (j-1)th scanning cycles, and n is an integer of 2 or more and represents a total number of scanning cycles.

In some embodiments, the method further includes determining that the scale formation occurs in the fluid mixture when the TSI value exceeds a predetermined threshold value over a defined time window.

In some embodiments, the method further includes determining that the scale formation occurs in the fluid mixture when the TSI value exceeds 1 over an initial five-hour time of the mixing.

In some embodiments, the method further includes calculating an average of the light transmission values at a bottom portion of the fluid mixture as a function of time. The bottom portion has a bottom height along the height direction, which is 20% or less of a height of the fluid mixture along the height direction.

In some embodiments, the method further includes determining that the scale formation occurs in the fluid mixture when the average of the light transmission values at the bottom portion of the fluid mixture decreases by at least 10% during an initial five-hour time of the mixing.

In some embodiments, the method further includes determining that the scale formation occurs in the fluid mixture when the average of the light transmission values at the bottom portion of the fluid mixture decreases by at least 2% per hour.

In some embodiments, the light transmission values as a function of the vertical position have a peak value and a plateau value, and the peak value is larger than the plateau value during an initial stage of the mixing.

In some embodiments, the method further includes monitoring the peak value and the plateau value during the mixing, and determining that the scale formation occurs in the fluid mixture when the peak value first equals the plateau value.

In some embodiments, the first solution includes 5-35 g/L of Na⁺, 10-80 g/L of Cl⁻, 0.1-1.2 g/L of Ca²⁺, 1-6 g/L of Mg²⁺, 0.05-0.50 g/L of HCO₃⁻, and 3-15 g/L of SO₄²⁻. The second solution includes 30-60 g/L of Na⁺, 100-300 g/L of Cl⁻, 15-25 g/L of Ca²⁺, 1-8 g/L of Mg²⁺, 0.05-0.50 g/L of HCO₃^{<}, and 0.01-0.50 g/L of SO₄²⁻.

In some embodiments, the first solution includes 18.29 g/L of Na⁺, 39.14 g/L of Cl⁻, 0.58 g/L of Ca²⁺, 2.88 g/L of Mg²⁺, 0.12 g/L of HCO₃⁻, and 4.29 g/L of SO₄²⁻. The second solution includes 59.48 g/L of Na⁺, 151.02 g/L of Cl⁻, 17.02 g/L of Ca²⁺, 3.33 g/L of Mg²⁺, 0.35 g/L of HCO₃⁻, and 0.35 g/L of SO₄²⁻.

In some embodiments, the first solution has a first total dissolved solid (TDS) concentration that is 50% or less of a second TDS concentration of the second solution, the first TDS concentration is 30,000-130,000 ppm, and the second TDS concentration is 150,000-400,000 ppm.

In some embodiments, the first TDS concentration is 65,000 ppm, and the second TDS concentration is 233,000 ppm.

In some embodiments, the scale formation occurs at least as a result of precipitation of CaSO₄.

In some embodiments, the scanning is executed for a scanning duration of at least 5 hours at a scanning interval of at most 10 minutes.

In some embodiments, the scanning duration is 12 hours to 48 hours, and the scanning interval is 5 seconds to 1 minute.

In some embodiments, the first solution is sea water, and the second solution is formation water present in an underground formation, the method further includes recovering hydrocarbons from the underground formation, which includes injecting the sea water into the underground formation so that the sea water and the formation water present in the underground formation mix with each other to form the fluid mixture.

In some embodiments, determining in real time whether the scale formation occurs in the fluid mixture based on the light transmission values during the recovering.

In some embodiments, the method further includes installing a light source and a light detector adjacent to a production well for the underground formation, and moving the light source and the light detector along a bottom portion of the production well in a height direction of the production well during the scanning.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings.

FIG. 1A illustrates an exemplary flow chart depicting a method for detecting and monitoring scale formation in a fluid mixture, according to certain embodiments.

FIG. 1B illustrates an experimental setup including a controlled laboratory apparatus configured to simulate the mixing of sea water (SW) and formation water (FW) under conditions representative of oil field environments, according to certain embodiments.

FIG. 2A illustrates a time-dependent transmission profile after a specified duration for a SW-FW 1:1 ratio mixture, according to certain embodiments.

FIG. 2B illustrates a time-dependent transmission profile after a specified duration for a SW-FW 3:2 ratio mixture, according to certain embodiments.

FIG. 3A illustrates a time-dependent transmission profile recorded after 48 hours for a SW-FW 1:4 ratio mixture, according to certain embodiments.

FIG. 3B illustrates a time-dependent transmission profile recorded after 48 hours for a SW-FW 3:7 ratio mixture, according to certain embodiments.

FIG. 3C illustrates a time-dependent transmission profile recorded after 48 hours for a SW-FW 2:3 ratio mixture, according to certain embodiments.

FIG. 3D illustrates a time-dependent transmission profile recorded after 48 hours for a SW-FW 7:3 ratio mixture, according to certain embodiments.

FIG. 3E illustrates a time-dependent transmission profile recorded after 48 hours for a SW-FW 4:1 ratio mixture, according to certain embodiments.

FIG. 4 illustrates a graph depicting transmission intensity values corresponding to each sea water and formation water mixture ratio, according to certain embodiments.

FIG. 5 illustrates a graph depicting transmission stability index (TSI) derived from the light transmission data of various brine ratios, according to certain embodiments.

FIG. 6A illustrates an optical image depicting initial condition of the brine sample prior to any mixing, according to certain embodiments.

FIG. 6B illustrates an optical image depicting the sample image after 48 hours for the SW-FW 1:1 ratio, according to certain embodiments.

FIG. 6C illustrates an optical image depicting sample image after 48 hours for the SW-FW 3:2 ratio, according to certain embodiments.

FIG. 6D illustrates an optical image depicting sample image after 48 hours for the SW-FW 1:4 ratio, according to certain embodiments.

FIG. 6E illustrates an optical image depicting sample image after 48 hours for the SW-FW 3:7 ratio, according to certain embodiments.

FIG. 6F illustrates an optical image depicting sample image after 48 hours for the SW-FW 2:3 ratio, according to certain embodiments.

FIG. 6G illustrates an optical image depicting sample image after 48 hours for the SW-FW 7:3 ratio, according to certain embodiments.

FIG. 6H illustrates an optical image depicting sample image after 48 hours for the SW-FW 4:1 ratio, according to certain embodiments.

FIG. 7A illustrates a position within the sample vial where precipitation occurred for a SW-FW 50:50 ratio, highlighting the region between 0 mm and 7 mm from the bottom of the vial, according to certain embodiments.

FIG. 7B illustrates the position within the sample vial where precipitation occurred for a SW-FW 60:40 ratio, highlighting the region between 0 mm and 6 mm from the bottom of the vial, according to certain embodiments.

FIG. 8 illustrates the transmission intensity profile recorded in the region between 0 mm and 7 mm for the SW-FW 50:50 ratio and between 0 mm and 6 mm for the SW-FW 60:40 ratio, according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, reference numerals designate identical or corresponding parts throughout the several views. Further, as used herein, the words ‘a,’ ‘an’ and the like generally carry a meaning of ‘one or more,’ unless stated otherwise.

Furthermore, the terms ‘approximately’, approximate’, ‘about’, and similar terms generally refer to ranges that include the identified value within a margin of 20%, 10%, or preferably 5%, and any values therebetween.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

As used herein, the term ‘production well’ refers to a borehole drilled into the earth to extract oil, gas, or geothermal energy.

As used herein, the term ‘hydrocarbons’ refers to organic compounds composed of hydrogen and carbon atoms, typically found in fossil fuels such as oil and natural gas.

As used herein, the term ‘scale formation’ refers to the precipitation and accumulation of mineral deposits, such as calcium carbonate or barium sulfate, on surfaces within equipment or pipelines, often due to changes in temperature, pressure, pH and/or a liquid (mixture) composition from which the scale forms.

As used herein, the term ‘total dissolved solids (TDS) concentration’ refers to the measure of the combined content of all inorganic and organic substances dissolved in water, expressed in milligrams per litre (mg/L).

As used herein, the term ‘peak value’ refers to the maximum point or level reached by a variable or signal during a specified period.

As used herein, the term ‘plateau value’ refers to a stable or constant level that a variable or signal reaches after an initial rise or change.

As used herein, the term ‘light transmission values’ refers to the percentage of light that passes through a sample or medium, indicating its transparency or clarity.

As used herein, the term ‘transmission stability index (TSI)’ refers to a numerical value that quantifies the consistency of light transmission through a medium over time or under varying conditions.

As used herein, the term ‘threshold value’ refers to the specific point or level at which a particular effect, response, or phenomenon begins to occur or be observed.

As used herein, the term ‘defined time window’ refers to a specified period during which certain conditions or activities are expected to occur or be monitored.

As used herein, the term ‘scanning cycle’ refers to one complete pass of a measurement device from the bottom to the top (or vice versa) of the fluid column to capture light transmission data across the height axis.

As used herein, the term ‘transmission profile’ describes how a sample allows light to pass through the sample. A ‘transmission profile’ is often expressed as a percentage, indicating how much light is transmitted relative to the amount of light that hits or is incident on the sample's surface. A ‘transmission profile’ can be specific to one particular wavelength or multiple wavelengths such as the entire spectrum of visible light. In the case of multiple wavelengths, a ‘transmission profile’ can be obtained by averaging over the multiple wavelengths. For example, a ‘transmission profile’ can refer to a spatial distribution of light transmission intensity values measured at discrete height intervals at a specific time point.

Aspects of the present disclosure are directed to a method for real-time monitoring of scale formation in fluid mixtures, particularly in oil and gas operations. This method addresses challenges posed by traditional monitoring techniques that often fail to detect early-stage scale formation due to variations in temperature, pressure, and water chemistry. By employing a turbidity scanner, the method enables continuous, non-invasive assessment of fluid turbidity and particulate changes, providing valuable insights into the interaction between sea water and formation water under controlled conditions. The method's sensitivity facilitates immediate operational adjustments to prevent scale accumulation, thereby enhancing flow assurance and enhancing water management strategies.

In addition, the method(s) as described hereinafter permits quantification of scale kinetics by correlating transmission variability with particle nucleation and growth rates. The aforementioned quantification may guide inhibitor dosing strategies and help identify supersaturation thresholds in field conditions. The disclosed method may be adapted to offshore rigs and subsea environments using fiber optic arrays integrated into production tubing or downhole tools. In some implementations, a turbidity scanner may be coupled with one or more spectroscopic tools such as, but not limited to, Raman and UV-Vis, in order to distinguish scale types based on chemical composition of the precipitates. Furthermore, anomalies in light transmission values at fixed positions over time may be used to construct spatiotemporal heat maps of scale intensity, offering visual feedback for field operators.

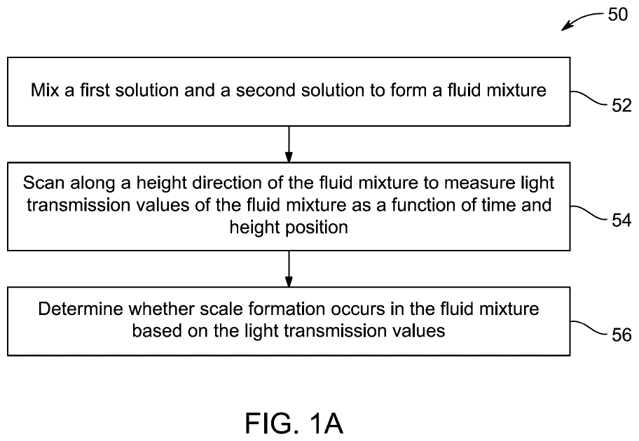

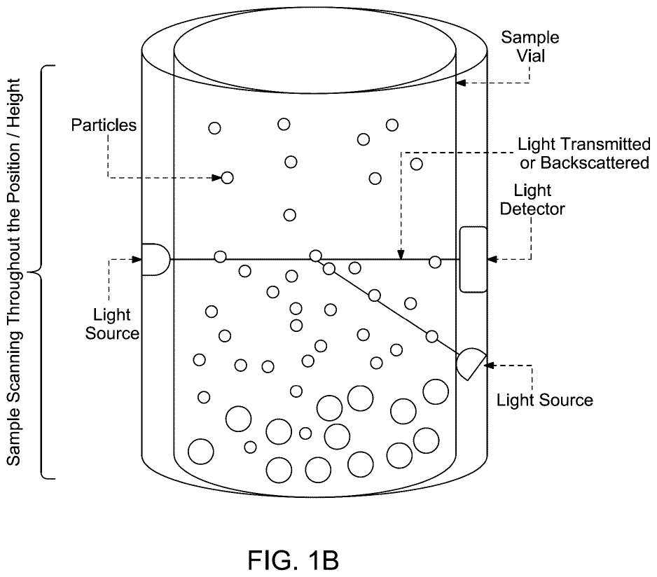

FIG. 1A illustrates a flow chart of a method 50 for detecting and monitoring scale formation in a fluid mixture. The scale formation occurs at least as a result of precipitation of CaSO₄. In oil and gas operations, scale formation is a common issue resulting from various factors beyond just calcium sulfate precipitation. Mixing incompatible waters, such as formation water with injected sea water or other brines, can lead to supersaturation and the precipitation of scales such as barium sulfate or strontium sulfate [Budiman, O., and Alajmei, S. Seawater-Based Fracturing Fluid: A Review. ACS Omega, 2023, 8, 44, 41022-41038, incorporated herein by reference in its entirety]. Pressure and temperature changes during fluid ascent can reduce mineral solubility, causing precipitation. pH variations also influence solubility; for example, an increase in pH can cause some minerals to precipitate. Evaporation during hydrocarbon production can concentrate dissolved salts beyond their solubility limits, resulting in scale deposition. Chemical reactions, such as the interaction of injected chemicals with formation water, can alter mineral solubility and promote scale formation. For instance, the use of sodium hydroxide in alkaline flooding can lead to the precipitation of silicate scales. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes mixing a first solution and a second solution to form the fluid mixture. The first solution has a first Ca²⁺ concentration that is 10% or less of a second Ca²⁺ concentration of the second solution, and a first SO₄²⁻ concentration that is 1000% or more of a second SO₄²⁻ concentration of the second solution. In some embodiments, the first solution is sea water, and the second solution is formation water obtained from an underground formation. The underground formation may include, but is not limited to, a depleted oil reservoir, a depleted gas reservoir, a sour reservoir, a hydrocarbon-bearing subterranean formation, a saline formation, or an un-minable coal bed.

In some embodiments, the first solution includes 5-35 g/L of Na⁺, 10-80 g/L of Cl⁻, 0.1-1.2 g/L of Ca²⁺, 1-6 g/L of Mg²⁺, 0.05-0.50 g/L of HCO₃⁻, and 3-15 g/L of SO₄²⁻. The first solution includes 5-35 grams per liter (g/L) of Na⁺, preferably 5.5-34.5 g/L, preferably 6-34 g/L, preferably 6.5-33.5 g/L, preferably 7-33 g/L, preferably 7.5-32.5 g/L, preferably 8-32 g/L, preferably 8.5-31.5 g/L, preferably 9-31 g/L, preferably 9.5-30.5 g/L, preferably 10-30 g/L, preferably 10.5-29.5 g/L, preferably 11-29 g/L, preferably 11.5-28.5 g/L, preferably 12-28 g/L, preferably 12.5-27.5 g/L, preferably 13-27 g/L, preferably 13.5-26.5 g/L, preferably 14-26 g/L, preferably 14.5-25.5 g/L, preferably 15-25 g/L, preferably 15.5-24.5 g/L, preferably 16-24 g/L, preferably 16.5-23.5 g/L, preferably 17-23 g/L, preferably 17.5-22.5 g/L, preferably 18-22 g/L of Na⁺. The first solution includes 10-80 g/L of Cl⁻, preferably 10.5-79.5 g/L, preferably 11-79 g/L, preferably 11.5-78.5 g/L, preferably 12-78 g/L, preferably 12.5-77.5 g/L, preferably 13-77 g/L, preferably 13.5-76.5 g/L, preferably 14-76 g/L, preferably 14.5-75.5 g/L, preferably 15-75 g/L, preferably 15.5-74.5 g/L, preferably 16-74 g/L, preferably 16.5-73.5 g/L, preferably 17-73 g/L, preferably 17.5-72.5 g/L, preferably 18-72 g/L, preferably 18.5-71.5 g/L, preferably 19-71 g/L, preferably 19.5-70.5 g/L, preferably 20-70 g/L, preferably 20.5-69.5 g/L, preferably 21-69 g/L, preferably 21.5-68.5 g/L, preferably 22-68 g/L, preferably 22.5-67.5 g/L, preferably 23-67 g/L, preferably 23.5-66.5 g/L, preferably 24-66 g/L, preferably 24.5-65.5 g/L, preferably 25-65 g/L, preferably 25.5-64.5 g/L, preferably 26-64 g/L, preferably 26.5-63.5 g/L, preferably 27-63 g/L, preferably 27.5-62.5 g/L, preferably 28-62 g/L, preferably 28.5-61.5 g/L, preferably 29-61 g/L, preferably 29.5-60.5 g/L, preferably 30-60 g/L, preferably 30.5-59.5 g/L, preferably 31-59 g/L, preferably 31.5-58.5 g/L, preferably 32-58 g/L, preferably 32.5-57.5 g/L, preferably 33-57 g/L, preferably 33.5-56.5 g/L, preferably 34-56 g/L, preferably 34.5-55.5 g/L, preferably 35-55 g/L, preferably 35.5-54.5 g/L, preferably 36-54 g/L, preferably 36.5-53.5 g/L, preferably 37-53 g/L, preferably 37.5-52.5 g/L, preferably 38-52 g/L, preferably 38.5-51.5 g/L, preferably 39-51 g/L of Cl⁻. The first solution includes 0.1-1.2 g/L of Ca²⁺, preferably 0.15-1.15 g/L, preferably 0.2-1.1 g/L, preferably 0.25-1.05 g/L, preferably 0.3-1.0 g/L, preferably 0.35-0.95 g/L, preferably 0.4-0.9 g/L, preferably 0.45-0.85 g/L, preferably 0.5-0.8 g/L, preferably 0.55-0.75 g/L of Ca²⁺. The first solution includes 1-6 g/L of Mg²⁺, preferably 1.5-5.5 g/L, preferably 2-5 g/L, preferably 2.5-4.5 g/L of Mg²⁺. The first solution includes 0.05-0.50 g/L of HCO₃⁻, preferably 0.10-0.45 g/L, preferably 0.15-0.40 g/L, preferably 0.20-0.35 g/L of HCO₃⁻. The first solution includes 3-15 g/L of SO₄²⁻, preferably 3.5-14.5 g/L, preferably 4-14 g/L, preferably 5-12 g/L, preferably 7-10 g/L of SO₄²⁻. In a preferred embodiment, the first solution includes 18.29 g/L of Na⁺, 39.14 g/L of Cl⁻, 0.58 g/L of Ca²⁺, 2.88 g/L of Mg²⁺, 0.12 g/L of HCO₃⁻, and 4.29 g/L of SO₄²⁻.

In some embodiments, the second solution includes 30-60 g/L of Na⁺, 100-300 g/L of Cl⁻, 15-25 g/L of Ca²⁺, 1-8 g/L of Mg²⁺, 0.05-0.50 g/L of HCO₃⁻, and 0.01-0.50 g/L of SO₄²⁻. The second solution includes 30-60 g/L of Na⁺, preferably 35-55 g/L, preferably 40-50 g/L, preferably 42-47 g/L of Na⁺. The second solution includes 100-300 g/L of Cl⁻, preferably 110-290 g/L, preferably 120-280 g/L, preferably 130-270 g/L, preferably 140-260 g/L, preferably 150-250 g/L; The second solution includes 15-25 g/L of Ca²⁺, preferably 15.5-24.5 g/L, preferably 16-24 g/L, preferably 16.5-23.5 g/L, preferably 17-23 g/L of Ca²⁺. The second solution includes 1-8 g/L of Mg²⁺, preferably 1.5-7.5 g/L, preferably 2-7 g/L, preferably 2.5-6.5 g/L, preferably 3-6 g/L, preferably 4-5 g/L of Mg²⁺. The second solution includes 0.05-0.50 g/L of HCO₃⁻, preferably 0.10-0.45 g/L, preferably 0.15-0.40 g/L, preferably 0.20-0.36 g/L of HCO₃. The second solution includes 0.01-0.50 g/L of SO₄²⁻, preferably 0.02-0.48 g/L, preferably 0.03-0.46 g/L, preferably 0.04-0.44 g/L, preferably 0.05-0.42 g/L, preferably 0.06-0.40 g/L, preferably 0.07-0.38 g/L, preferably 0.08-0.36 g/L, preferably 0.10-0.30 g/L, preferably 0.15-0.25 g/L, preferably 0.17-0.23 g/L of SO₄²⁻. In a preferred embodiment, the second solution includes 59.48 g/L of Na⁺, 151.02 g/L of Cl⁻, 17.02 g/L of Ca²⁺, 3.33 g/L of Mg²⁺, 0.35 g/L of HCO₃⁻, and 0.35 g/L of SO₄²⁻.

The first solution can have a first total dissolved solid (TDS) concentration that is 50% or less of a second TDS concentration of the second solution. The first TDS concentration can be 30,000-130,000 parts per million (ppm), preferably 35,000-125,000 ppm, preferably 40,000-120,000 ppm, preferably 45,000-115,000 ppm, preferably 50,000-110,000 ppm, preferably 55,000-105,000 ppm, preferably 60,000-100,000 ppm, preferably 62,000-95,000, preferably 70,000-90,000 preferably 75,000-85,000. The second TDS concentration can be 150,000-400,000 ppm, preferably 160,000-390,000 ppm, preferably 170,000-380,000 ppm, preferably 180,000-370,000 ppm, preferably 190,000-360,000 ppm, preferably 200,000-350,000 ppm, preferably 210,000-340,000 ppm, preferably 220,000-330,000 ppm, preferably 230,000-320,000 ppm, preferably 250,000-300,000 ppm, preferably 270,000-280,000 ppm. In a preferred embodiment, the first TDS concentration is 65,000 ppm while the second TDS concentration is 233,000 ppm.

At step 54, the method 50 includes scanning along a height direction of the fluid mixture to measure light transmission values of the fluid mixture as a function of time and height position. The scanning can for example be executed for a scanning duration of at least 5 hours at a scanning interval of at most 10 minutes. The scanning duration can be 12 hours to 48 hours, preferably 12.5 to 47.5 hours, preferably 13 to 47 hours, preferably 13.5 to 46.5 hours, preferably 14 to 46 hours, preferably 14.5 to 45.5 hours, preferably 15 to 45 hours, preferably 15.5 to 44.5 hours, preferably 16 to 44 hours, preferably 16.5 to 43.5 hours, preferably 17 to 43 hours, preferably 17.5 to 42.5 hours, preferably 18 to 42 hours, preferably 18.5 to 41.5 hours, preferably 19 to 41 hours, preferably 19.5 to 40.5 hours, preferably 20 to 40 hours, preferably 20.5 to 39.5 hours, preferably 21 to 39 hours, preferably 21.5 to 38.5 hours, preferably 22 to 38 hours, preferably 22.5 to 37.5 hours, preferably 23 to 37 hours, preferably 23.5 to 36.5 hours, preferably 24 to 36 hours, preferably 28 to 32 hours. The scanning interval can be 1 second to 9 minutes, preferably 5 seconds to 8 minutes, preferably 10 seconds to 5 minutes, preferably 10 seconds to 3 minutes, preferably 20 seconds to 2 minutes, preferably 30 seconds to 1 minute.

At step 56, the method 50 includes determining whether scale formation occurs in the fluid mixture based on the light transmission values.

In some embodiments, the method 50 further includes calculating a transmission stability index (TSI) value based on a difference between consecutive transmission profiles of the light transmission values along the height direction of the fluid mixture.

$$SI ( {t}_{n} ) = {\sum }_{j = 1}^{n} \frac{{\sum }_{{z}_{min}}^{{z}_{max}} \vert I ( {t}_{j} , {z}_{i} ) - I ( {t}_{j - 1} , {z}_{i} ) \vert}{{z}_{n}}$$

where: SI(tₙ) is the TSI value at time of tₙ, I(tⱼ, zᵢ) is transmitted light intensity at time of tⱼ and position of zᵢ, I(tⱼ₋₁, zᵢ) is transmitted light intensity at time of tⱼ₋₁ and position of zᵢ, zₘₐₓ is a highest height position along the height direction of the fluid mixture, zₘᵢₙ is a lowest height position along the height direction of the fluid mixture, zₙ=zₘₐₓ−zₘᵢₙ and represents a height of the fluid mixture, tₙ, tⱼ and tⱼ₋₁ respectively represent time during nth, jth and (j-1)th scanning cycles, and n is an integer of 2 or more and represents a total number of scanning cycles.

In some embodiments, the method 50 further includes determining that the scale formation occurs in the fluid mixture when the TSI value exceeds a predetermined threshold value over a defined time window. The method 50 further includes determining that the scale formation occurs in the fluid mixture when the TSI value exceeds 1 over an initial five-hour time of the mixing. In some embodiments, a series of TSI values are recorded over successive scanning cycles, and a trend analysis is performed to establish persistent instability in transmission values. Such analysis enables characterization of progressive scale growth kinetics.

In some embodiments, the method 50 further includes calculating an average of the light transmission values at a bottom portion of the fluid mixture as a function of time. The bottom portion has a bottom height along the height direction, which is 20% or less of a height of the fluid mixture along the height direction. The method 50 further includes determining that the scale formation occurs in the fluid mixture when the average of the light transmission values at the bottom portion of the fluid mixture decreases by at least 10% during an initial five-hour time of the mixing. The method 50 further includes determining that the scale formation occurs in the fluid mixture when the average of the light transmission values at the bottom portion of the fluid mixture decreases by at least 2% per hour. In another embodiment, a cumulative turbidity score is generated for the bottom portion using transmission gradients, allowing temporal profiling of particle accumulation.

The light transmission values as a function of the vertical position can have a peak value and a plateau value. The peak value may be larger than the plateau value during an initial stage of the mixing. The method 50 further includes monitoring the peak value and the plateau value during the mixing. The method 50 further includes determining that the scale formation occurs in the fluid mixture when the peak value first equals the plateau value. In some implementations, differential analysis between peak and plateau values is plotted in real time to monitor convergence trends indicative of colloidal settling or crystallization fronts.

In some embodiments, the method 50 further includes recovering hydrocarbons from the underground formation, which includes injecting the sea water into the underground formation so that the sea water and the formation water present or naturally occurring in the underground formation mix with each other to form the fluid mixture. The sea water may be injected into the subterranean geological formation through a wellbore. In one or more embodiments, the wellbore may be present in at least one of an oil well, a gas well, a production well, an injection well, a naturally flowing well, an artificially lifted well, a high-temperature well, a steam-assisted gravity drainage well, a steam injector well, or a geothermal well. The wellbore may be formed in the subterranean geologic formation by known techniques. In one embodiment, the method 50 is implemented in offshore subsea operations, where sea water injection and in-line monitoring occur within riser assemblies or subsea manifolds.

The method 50 can further include determining in real time whether the scale formation occurs in the fluid mixture based on the light transmission values during the recovering. In one embodiment, time-resolved spectrophotometry is integrated to record transmission changes at multiple wavelengths, allowing scale speciation and mineral identification. In another embodiment, a machine learning model may be trained on historic transmission profiles to predict impending scale events based on real-time data inputs.

The method 50 can further include installing a light source and a light detector adjacent to a production well for the underground formation. In some embodiments, the light source may include, but is not limited to, such as tunable diode lasers (TDLS), incandescent tungsten-halogen lamps, light emitting diodes (LEDs), xenon flashtubes, optically powered light sources, laser diodes, high-intensity discharge (HID) lamps, fluorescent lamps, cap lamps, fiber optic light sources. In some embodiments, the light detector may include, but is not limited to photodetector modules, spectrometers, infrared detectors, LiDAR sensors, distributed acoustic sensing (DAS) systems, photodiodes, photomultiplier tubes (PMTs), phototransistors, photomultiplier tubes (PMTs), quantum dot photodetectors. In some embodiments, fiber optic sensors are embedded in downhole tubing to create a distributed transmission map along the entire height of the wellbore.

The method 50 can further include moving the light source and the light detector along a bottom portion of the production well in a height direction of the production well during the scanning. In some embodiments, mechanisms such as remotely operated vehicles (ROVs), crawler mechanisms, track or rail systems, gantry systems, coiled tubing conveyance, capstan winches, hydraulic actuators, rotary actuators, linear actuators, programmable logic controllers (PLCs), microprocessors, feedback mechanisms, automated control systems, sensor integration, real-time monitoring systems, data acquisition systems (DAQs), wireless communication modules, surface control units, interface panels may be used for moving/synchronizing the light source and the light detector. In one embodiment, an automated extension such as a robotic arm may be mounted within the well casing enables programmed scanning trajectories, including vertical sweeps and rotational mapping. In another embodiment, scanning is synchronized with flow rate variations to map deposition behavior under dynamic production conditions.

In some embodiments, the fluid mixture may include any suitable additives. Exemplary additives include, but are not limited to, weighting agents, emulsifiers, viscosities, fluid-loss control agents, bridging agents, pH controlling agents, defoamers, clay stabilizers, anti-scalants, deflocculants, lubricants, gelling agents, corrosion inhibitors, rheology control modifiers or thinners, high temperature/high pressure control additives, acids, alkalinity agents, pH buffers, fluorides, gases, nitrogen, carbon dioxide, surface modifying agents, tackifying agents, foamers, catalysts, clay control agents, biocides, bactericides, friction reducers, antifoam agents, dispersants, flocculants, H₂S scavengers, CO₂ scavengers, oxygen scavengers, friction reducers, breakers, relative permeability modifiers, resins, particulate materials such as proppant particulates, wetting agents, coating enhancement agents, filter cake removal agents, odorants, shale stabilizers, or any combinations thereof. In one embodiment, one or more anti-scalants such as phosphonates or polyacrylate compounds are added, and an influence thereof on TSI values is tracked to evaluate additive effectiveness. In another embodiment, additive interactions are mapped by correlating TSI and bottom transmission trends under varying fluid chemistries.

### Examples

The following examples demonstrate a method for detecting and monitoring scale formation in a fluid mixture. The examples are provided solely for illustration and are not to be construed as limitations of the present disclosure, as many variations thereof are possible without departing from the spirit and scope of the present disclosure.

### Example 1: Methods and materials

The brines used in this study include sea water (SW) with a total dissolved solids (TDS) concentration of 65,000 ppm, and formation water (FW) with a TDS concentration of 233,000 ppm. These mixtures simulate conditions typical of oil recovery operations. Table 1 lists the ions present in the brines used for the experiments. The experiments occur at a constant temperature of 70° C. over a 48-hour period, with varying SW-FW ratios of 1:1, 2:3, 3:7, 1:4, 3:2, 7:3, and 4:1. The experiment matrix is summarized in Table 2. Turbidity scans monitor the rate of scale formation and detect its occurrence when mixing sea water (SW) with formation water (FW) at varying ratios. These scans measure the cloudiness or haziness in a fluid, which correlates directly to the amount of scale precipitating from the solution. As the mixing of SW and FW results in mineral scale formation, the turbidity of the solution increases. By continuously measuring the turbidity over the 48-hour observation period, the progression of scale formation is tracked in real time. This method enables the determination of the precipitation rate at which scale forms under different SW-FW mixing ratios.

TABLE 1
Sea water and formation water composition

| | Sea water | Formation |
|---|---|---|
| Ions | (g/l) | water (g/l) |
| Na²⁺ | 18.29 | 59.48 |
| Cl⁻ | 39.14 | 151.02 |
| Ca²⁺ | 0.58 | 17.02 |
| Mg²⁺ | 2.88 | 3.33 |
| HCO₃⁻ | 0.12 | 0.35 |
| SO₄²⁻ | 4.29 | 0.35 |

TABLE 2
Experiment compositions and conditions

Ratio

| Sea water | Formation water | Temperature | Time |
|---|---|---|---|
| 1 | 4 | 70° C. | 48 h |
| 3 | 7 | | |
| 2 | 3 | | |
| 1 | 1 | | |
| 3 | 2 | | |
| 7 | 3 | | |
| 4 | 1 | | |

Based on the SW and FW compositions, the possible scales that could form include CaSO₄ and NaCl. The precipitation of NaCl primarily occurs as water evaporates into the gas phase, often triggered by a decrease in pressure, especially in wells with a low water-to-oil ratio, and is typically detected in HPHT (High Pressure High Temperature) gas wells [Oscar Vazquez, Modelling Oilfield Scale Squeeze Treatments: From Core to Reservoir, Springer, 2023, 2509-3134, incorporated herein by reference in its entirety]. Sulfate scale deposition arises from the mixing of incompatible brines, with the potential for scale formation being influenced by the fraction of sea water. Typically, the highest potential for scale formation is observed at a sea water fraction of around 50%, where there is a balance between divalent cations and sulfate ions. Sulfate scales, in this case, CaSO₄, could form when the following equilibrium moves to the right:

$${Ca}_{( aq )}^{2 +} + {SO}_{4 ( aq )}^{2 -} ↔ {CaSO}_{4 ( s )}$$

### Example 2: Turbidity Scan Technique

Turbidity scanning is a non-invasive analytical technique to assess colloidal systems' physical stability, such as emulsions, suspensions, and dispersions (Polowczyk et al., 2015; Sun et al., 2019, incorporated herein by reference in their entirety). Once the sample is placed in the turbidity scanner, the instrument is calibrated using a reference liquid, a standard solution with known turbidity. A monochromatic light source illuminates the sample, often in the near-infrared range. Two detectors collect light data: transmitted light and backscattered light. These signals are measured along the vertical axis of the sample, producing detailed transmission and backscattering profiles. Scans are repeated at fixed intervals to monitor physical changes. The system generates time-resolved optical profiles, which are used to calculate a Stability Index (SI).

This method was used to monitor real-time precipitation behavior in mixtures of seawater (SW) and formation water (FW). The experiments were designed to simulate oilfield conditions and evaluate the start and development of scale formation by continuously measuring light transmission through the sample. SW and FW were combined in various ratios and maintained at 70° C. for 48 hours in a transparent 22 mL vial, which was scanned every 15 seconds along its 50 mm height to measure transmitted and backscattered light intensity. The Transmission Stability Index (Tr-SI) quantified changes in turbidity over time by calculating differences in transmission profiles between scans. Significant increases in Tr-SI and drops in transmission indicated rapid scale formation, while stable transmission and low Tr-SI suggested minimal precipitation. This procedure aligns with ASTM D1889 and EPA Method 180.1, using approximately 880 nm light to assess turbidity.

This technique provides a detailed characterization of the sample, offering insights into several parameters, such as changes in average particle or droplet size, the relative size distribution, and the effective concentration of the dispersed phases. Additionally, turbidity scanning allows for the evaluation of the time and temperature stability of dispersions and emulsions. The turbidity scan includes an option to calculate the transmission intensity over time and stability index (Tr-SI or Bs-SI), which is based on transmitted or backscattered light, respectively. The stability index (SI) can be determined either as differences or homogeneity. In this study, the Transmission Stability Index (TSI) based on the differences in transmission light is used to calculate the transmission intensity and stability index. The method as described herein calculates the successive differences between consecutive profiles at times tⱼ and tⱼ₋₁, providing a measure of how much the sample's turbidity changes over time.

$$SI ( {t}_{n} ) = {\sum }_{j = 1}^{n} \frac{{\sum }_{{z}_{min}}^{{z}_{max}} \vert I ( {t}_{j} , {z}_{i} ) - I ( {t}_{j - 1} , {z}_{i} ) \vert}{{z}_{n}} \qquad ( A )$$

SI(tₙ) is the TSI value at time of tₙ, I(tⱼ, zᵢ) is transmitted light intensity at time of tⱼ and position of zᵢ, I(tⱼ₋₁, zᵢ) is transmitted light intensity at time of tⱼ₋₁ and position of zᵢ, zₘₐₓ is a highest height position along the height direction of the fluid mixture, zₘᵢₙ is a lowest height position along the height direction of the fluid mixture, zₙ=zₘₐₓ−zₘᵢₙ and represents a height of the fluid mixture, tₙ, tⱼ and tⱼ₋₁ respectively represent time during nth, jth and (j-1)th scanning cycles, and n is an integer of 2 or more and represents a total number of scanning cycles.

The equation (A) is derived based on Lambert's law as follows. In particular, when a beam of light travels through a medium, an intensity of the beam of light is reduced due to scattering and absorption by suspended particles. Lambert's law governs this attenuation of the incident light intensity.

$${I}_{T} ( t , z ) = {I}_{o} {e}^{- α ( t , z ) l} \qquad ( 1 )$$

where I_{T}(t, z) is the transmitted light intensity at time t, position z, Iₒ is the incident light transmission, a (t, z) is the absorption plus scattering coefficient, which varies with time and height due to changing turbidity, and l is the optical path length between source and sensor.

As mineral scale forms in the brine system such as BaSO₄, SrSO₄ and CaCO₃, the number and size of suspended particles increase, leading to a higher attenuation coefficient α(t, z), which manifests as a measurable decrease in transmitted light intensity I_{T}(t, z), which serves as a direct optical indicator of nucleation, growth, and settling processes. By monitoring I_{T}(t, z) continuously across both spatial and temporal domains, the system provides detailed insights into the kinetics of scale formation. The reduction in transmission reflects the onset and progression of turbidity associated with particle generation, offering a dynamic, non-invasive means of tracking mineral precipitation in real time.

At any fixed height z, changes in transmitted intensity over time reflect localized turbidity evolution. This effect may be represented by equation (2) as provided below:

$$\frac{\partial I ( t , z )}{\partial t} \qquad ( 2 )$$

A negative value indicates increasing turbidity (more scattering, which means more particle formation), while a positive value suggests dissolution or settling. In order to obtain a global measure of system-wide transmission instability, local derivative is integrated across the full vertical extent of the column, as provided in equation (3):

$$SI ( t ) = \frac{1}{{z}_{n}} {\int }_{{z}_{min}}^{{z}_{max}} \vert \frac{\partial I ( t , z )}{\partial t} \vert dz \qquad ( 3 )$$

where SI(t) is the transmission stability index and zₙ=zₘₐₓ−zₘᵢₙ is the total sample height. The absolute value ensures that both upward and downward fluctuations in intensity are captured. Equation (3) accounts for localized variations and generates a height-averaged index that increases during active precipitation periods and stabilizes when the system reaches equilibrium.

In real systems, particle precipitation may exhibit rapid changes and gradual transitions due to slow aggregation or delayed kinetics. In order to account for such behaviours, a second-order time derivative of transmission intensity is included, as depicted in equation (4) and equation (5):

$$\frac{d}{dt} SI ( t ) = \frac{d}{dt} \frac{1}{{z}_{n}} {\int }_{{z}_{min}}^{{z}_{max}} \vert \frac{\partial I ( t , z )}{\partial t} \vert dz \qquad ( 4 )$$

$$\frac{d}{dt} SI ( t ) = \frac{1}{{z}_{n}} {\int }_{{z}_{min}}^{{z}_{max}} [ \vert \frac{\partial I ( t , z )}{\partial t} \vert + \vert \frac{{\partial }^{2} I ( t , z )}{\partial {t}^{2}} \vert ] dz \qquad ( 5 )$$

where

$$\frac{{\partial }^{2} I ( t , z )}{\partial {t}^{2}}$$

captures acceleration or deceleration in turbidity change. Equation (4) and equation (5) allows Tr-SI to resolve sudden nucleation bursts and gradual aggregation or dispersion. Equations (3) and equation (5) may also be expressed in a discrete form, referred to as SI (tₙ) above in equation (A). While the present disclosure uses the simplified form for practical analysis, the complete derivation presented here provides a theoretical foundation to support modelling and simulation efforts.

### Example 3: Experimental Setup

The experimental setup, as shown in FIG. 1B, includes a controlled laboratory apparatus designed to simulate the mixing of sea water and formation water under conditions that mimic those found in oilfield environments. The temperature is set to 70° C., and the apparatus includes a light source that directs light into the sample vial containing the fluid to be measured, a sample vial, which holds the fluid sample with suspended particles and is typically transparent to allow the light to pass through, sample scanning, where the sample vial is scanned throughout its height to create a transmission position profile, providing a representative turbidity measurement. The apparatus further includes a detector, positioned opposite the light source, which detects two types of light intensities including transmitted intensity (the light that passes through the sample without being scattered by the particles) and backscattered intensity (the light scattered back by the suspended particles in the sample) and a change in intensities, where the difference between the light emitted by the source and the light received by the detector is measured to quantify the turbidity of the fluid. Higher turbidity results in less transmitted light and more backscattered light, while lower turbidity leads to more transmitted light and less backscattered light.

As light passes through the fluid sample, it interacts with the particles within the fluid. These particles scatter and absorb light, affecting the intensity of light that reaches the other side of the sample. The intensity of light that transmits through the sample is measured. The presence of particles within the fluid typically decreases the intensity of transmitted light due to scattering and absorption. The level of turbidity, or cloudiness, in the fluid is directly related to the intensity of the transmitted light. Higher turbidity levels result in less light being transmitted, while lower turbidity levels allow more light to pass through. The variations in scale formation observed across different sea water (SW)-formation water (FW) mixing ratios can be attributed to the principles of chemical equilibrium and solubility. In brine mixtures, the potential for scale formation is governed by the solubility product (Ksp) of the possible scales. When the ionic product of the dissolved ions exceeds the Ksp, precipitation occurs. The results of the experiment may be divided into two groups.

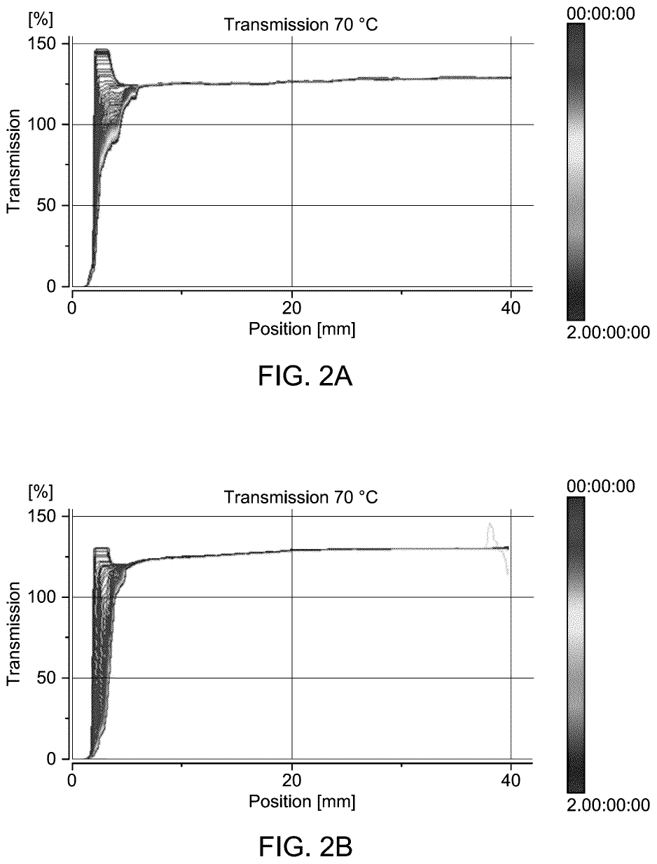

The first group of data, which includes the 1:1 and 3:2 ratios of SW to FW, demonstrated a pattern of high mineral scale precipitation. Turbidity scans for these ratios revealed a notable decrease in light transmission in the lower millimetre range (which corresponds to a bottom position of the bottle), as shown in FIGS. 2A-2B. Therefore, when SW and FW are mixed in the aforementioned proportions, the conditions are highly conducive to sulfate scale formation, possibly due to a balance of divalent cations and sulfate ions, which precipitate as sulfate scales. Additionally, the consistency of precipitation pattern across the mentioned ratios implied that there is a range around the equimolar point where the ionic balance remains conducive to scale formation. It may be due to the relative concentrations of ions that favour the formation of a particular scale type within this specific window of ratios. Similar results across these ratios indicate a region of the SW-FW mixing spectrum where scale formation is not only probable but also quite efficient, leading to a rapid decline in the transmission properties of the fluid and an increase in turbidity. Localized light attenuation as shown by initial highlighted area in FIGS. 2A-2B signifies strong light attenuation, corresponding to high turbidity or dense particle accumulation. Further, in ratios of 1:1 and 3:2, the concentration of divalent cations such as Ca²⁺ from formation water and sulfate ions from sea water is conducive for CaSO₄ scale precipitation, resulting in rapid and significant formation of solid particles. The solid particles settle due to gravity, accumulating at the bottom, which may lead to a localized drop in transmitted light detected by the turbidity scanner, hence the red zone in the lower millimetres.

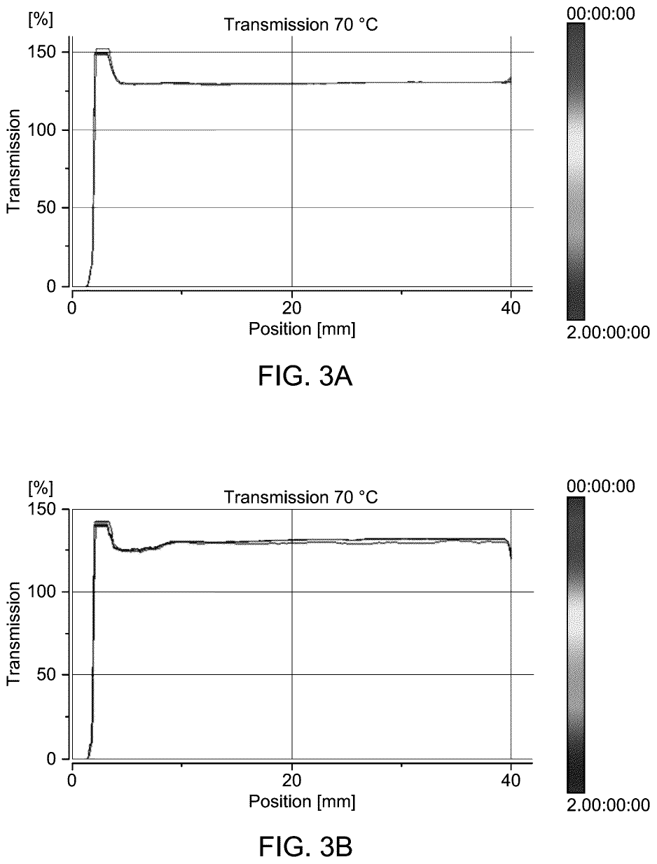

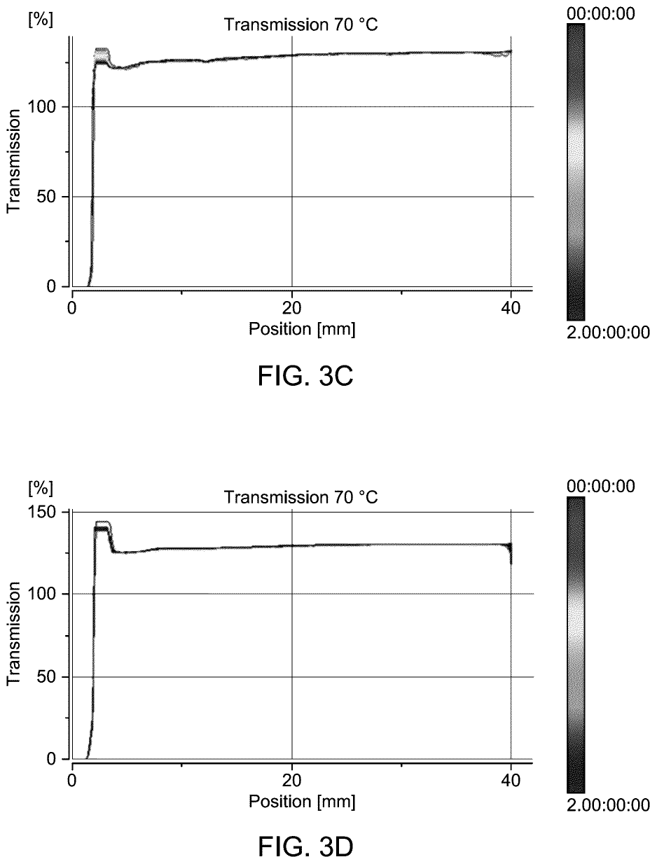

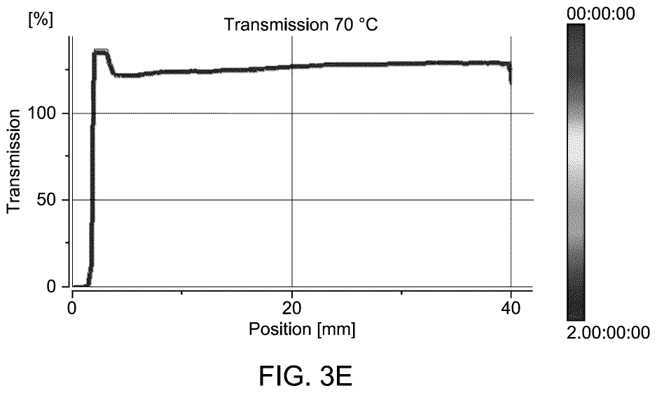

In contrast, the second group, which included the 1:4, 3:7, 2:3, 7:3, and 4:1 ratios, showed a different pattern where light transmission remains consistently high across the measurement range, indicating low turbidity. This pattern, as shown in FIGS. 3A-3E, implies a lower incidence of mineral scale precipitation. The ratios as mentioned herein suggest that the conditions are not as favourable for scale formation and/or that the resulting scales have higher solubility under the test conditions. The consistent light transmission observed suggests that these disproportionate ratios do not create a conducive environment for supersaturation and subsequent precipitation of sulfate scales.

At the mentioned ratios, particularly the 1:4, 3:7, and 2:3 mixtures where FW is dominant, the concentration of sulfate ions from SW may be too low to react extensively with the divalent cations such as calcium and magnesium from FW. The lower availability of sulfate ions may result in a sub-threshold concentration that may not favour the rapid formation of minerals. Conversely, in the 4:1 and 7:3 mixtures where SW is more prevalent, the excess of sulfate ions in relation to divalent cations might also suppress scale formation. The relative surplus of sulfate ions may lead to a scenario where many of these ions remain in solution rather than forming precipitates, potentially because not enough divalent cations are present to reach the Ksp for any potential scale mineral.

A noticeable difference between the first and second groups is the transmission level, which indicates the amount of scale formed. The first group shows a sharp decline in transmission at the outset, suggesting high turbidity due to significant scale formation or mineral precipitation. In contrast, the second group maintains high transmission, indicating lower turbidity and, consequently, less scale formation. The distinction implies that the precipitation rate of sulfate scale is highly dependent on the specific SW-FW mixing ratios, with the first group favouring scale formation much more than the second group. The occurrence of these patterns can be attributed to the ion concentration and the ionic balance in the brine mixtures. At approximately equal ratios (near the 50-50 mark), the ionic makeup is ideal for supersaturation and precipitation of scales, where SW ions meet FW ions in sufficient quantities to exceed solubility limits. As the balance shifts away from this midpoint, the possibility for the scales to form diminishes, possibly due to a lack of sufficient divalent cations or sulfate ions to reach the levels necessary for precipitation.

Further, transmission intensity provides a measure of the uniformity and consistency of turbidity of a fluid over time. If there is little to no change in the transmission intensity over time, it suggests that the fluid has a low particulate content. The aforesaid condition implies that the fluid is relatively clear and that solutes contained in the fluid are dissolved rather than precipitating out as discrete particles. Furthermore, an increase in the variability of transmission intensity over time indicates that the fluid is becoming more turbid, suggesting a rise in particulate content. In the context of mineral precipitation, the aforementioned variability signifies that minerals are coming out of solution to form solid particles.

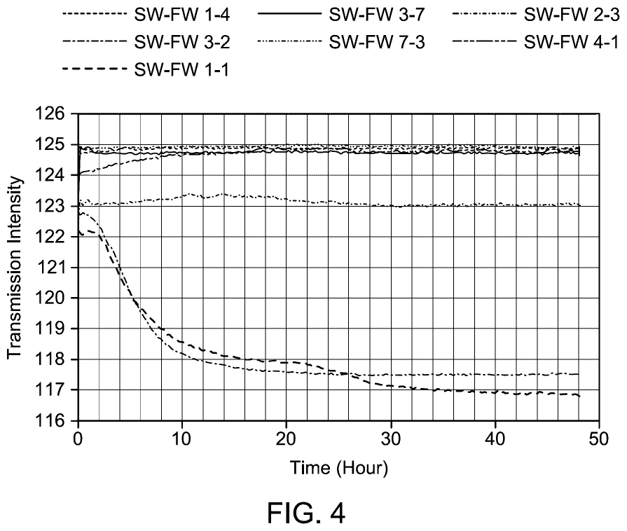

FIG. 4 shows the transmission intensity for each SW-FW ratio. The behaviour of each line suggests varying degrees of turbidity and, by implication, mineral precipitation in each fluid sample. A decreasing trend in the transmission value over time is a clear indicator that the fluid is becoming more turbid due to the precipitation of minerals. As these minerals form solid particles, they scatter and reflect light, reducing the amount of light that may pass directly through the fluid. The decrease in transmission intensity is a direct measure of the increase in particle concentration and size, signalling the progression of mineral precipitation.

The SW-FW 1:1 and 3:2 ratios show a sharp decrease in transmission intensity, dropping significantly within the first 10 hours. After this decrease, the transmission intensity levels off, indicating a substantial mineral precipitation event that stabilizes after the initial reaction. The SW-FW 1:4, 3:7, 7:3, and 4:1 ratios display stability with inconsequential fluctuation in transmission intensity, suggesting that clarity of the fluid remained consistent over time, with very few particles being formed or remaining in solution. While the 2:3 ratio showed a lower starting point compared to the 1:4, 3:7, 7:3, and 4:1 ratios but higher than the 1:1 and 3:2 ratios, it suggests that this ratio has a larger soluble particle concentration compared to the 1:4, 3:7, 7:3, and 4:1 ratios, but fewer particles than the 1:1 and 3:2 ratios. Over time, the 2:3 ratio showed only a slight change in transmission intensity and an early stabilization, implying of the absence of precipitation.

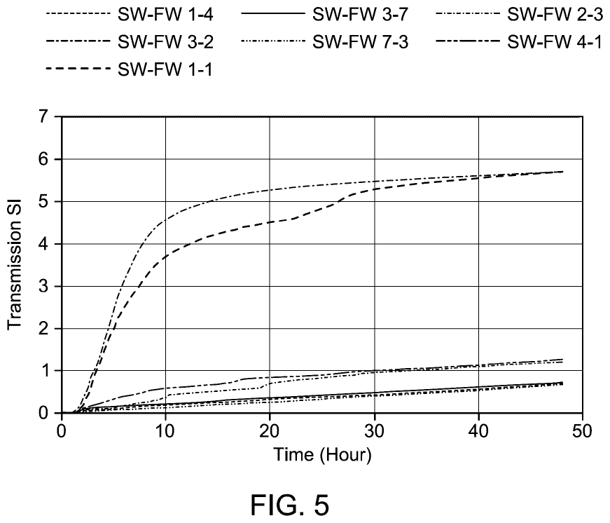

Transmission stability indices (TSI) as differences provide a quantifiable assessment of the stability of a fluid with respect to its turbidity over time. TSI as differences refers to the method of calculating stability by assessing the changes between different turbidity profiles at various time points. A fluid characterized by consistent transmission values over time, suggesting minimal changes in turbidity, will yield lower TSI values. This is indicative of a stable system with negligible scale formation. In contrast, a fluid undergoing scale formation will display significant changes in transmission, as detected by the successive transmission measurements, resulting in higher TSI values. The aforementioned dynamic assessment, as depicted in FIG. 5 for each SW-FW ratio, serves as a critical tool in discerning the temporal aspects of scale formation and stability in fluid systems.

For the SW-FW 1:4, 3:7, 2:3, 7:3, and 4:1 ratios, the transmission remains relatively flat and low over the 48-hour period, suggesting a stable mixture with inconsequential changes in turbidity and thus little or no scale formation. The stability may be due to an equilibrium state where the rate of scale formation is slow or negligible. In contrast, the SW-FW 1:1 and 3:2 ratios show a dramatic increase, with transmission values continuing to rise throughout the experiment. This suggests ongoing scale formation, possibly due to a continuous reaction between the ions in the SW and FW at these ratios. Overall, the graph reflects the dynamic nature of scale formation under different SW-FW mixing conditions, with varying degrees of reaction intensity and stability over time. The TSI provides a clear visualization of the time-dependent behavior of each mixture, highlighting the specific ratios that are more prone to scale formation and the effectiveness of the mixtures to reach a stable state after initial reactions.

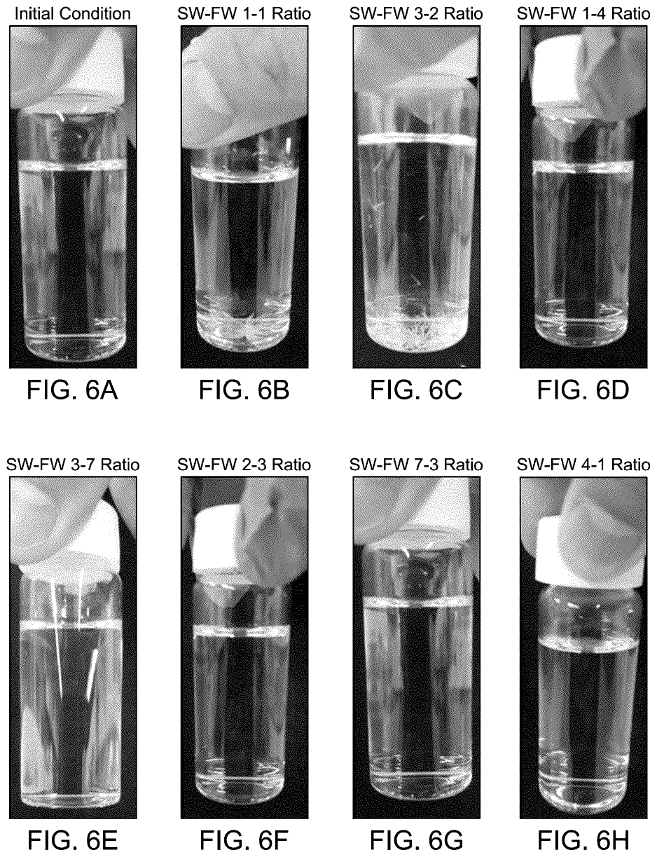

After 48 hours, a visual examination was performed to validate the results. The samples with unstable TSI and lower intensity ratios, specifically the 1:1 and 3:2 ratios, revealed presence of sharp, needle-like mineral formations. The findings are consistent with the higher turbidity and scale formation indicated by the TSI. In contrast, samples from the other ratios maintained clarity and appeared similar to their initial conditions, aligning with the stable TSI readings and suggesting minimal to no scale formation. FIGS. 6A-6H shows the sample conditions after 48 hours at 70° C.

Both temporal and spatial parameters influenced the precipitation process, and the governing equation accounted for variations along the z-axis over time. The precipitation behavior remained stochastic and occurred at multiple localized regions within the medium. The majority of precipitation was detected in the 0 to 6 mm depth range, while minor changes in transmission intensity were recorded in the upper regions. The presence of smaller suspended particles that did not readily settle was inferred from the observed upper-region transmission behavior. Further, optical transmission measurements from 0 to 40 mm across the vertical length of the vial containing the test sample were recorded. Precipitation primarily occurred at the bottom of the vial. The bottom-settling behavior confirmed the formation of discrete precipitates. Uniform transmission reduction throughout the vial was not observed, excluding incompatibility-induced haziness or uniformly suspended particulate matter. In the 50:50 ratio mixture, a marked reduction in transmitted light occurred within the 0 to 7 mm range, identifying the zone of highest precipitation intensity. Similarly, the 60:40 ratio mixture exhibited a transmission drop from 0 to 6 mm. Regions beyond the said intervals exhibited minimal changes in transmission.

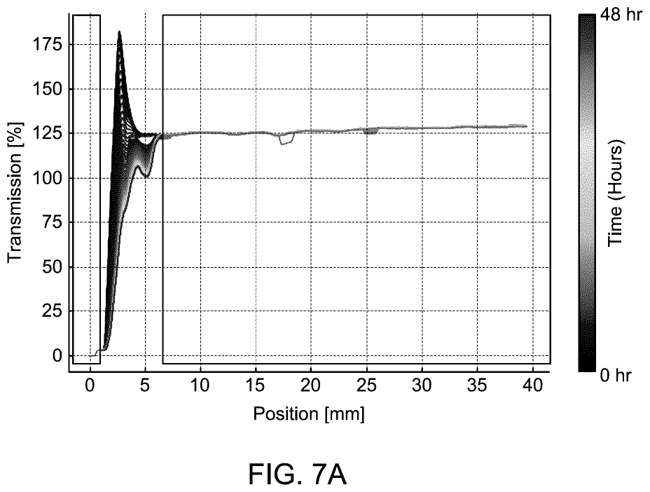

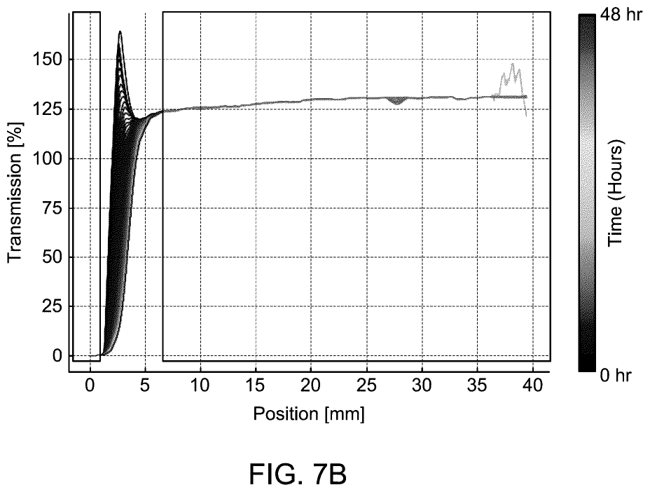

FIGS. 7A-7B show the specific intervals along the sample depth where transmission intensity significantly changed due to the precipitation, confirming spatial variation in deposition within the container, according to certain embodiments.

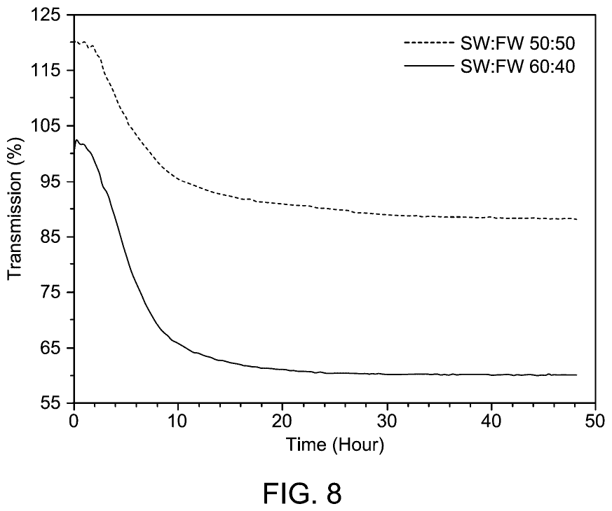

A differential stability profile was observed between two tested ratios of SW to FW including 60:40 and 50:50. The 60:40 ratio demonstrated reduced stability when compared to the 50:50 ratio. FIG. 8 shows the recorded transmission intensity (or transmission) across the sample height, according to certain embodiments. Spatially resolved turbidity profiling provided data on the location and magnitude of instability, enabling differentiation between zones containing suspended particulate matter and regions where precipitate had settled, according to certain embodiments.

Experimental conditions for FIG. 8 are as follows. (1) Equipment Calibration. Calibration was performed with no medium present between the light source and detector. The transmission reading was 100%, confirming that the system and calibration were functioning correctly. (2) Empty Vial. Introduce an empty vial into the optical path, which reduces the transmission to approximately 80%. This decrease is attributed to Fresnel reflections and refraction losses at the air-glass interfaces, which causes scattering and partial redirection of the incident light. (3) Vial Filled with Deionized (DI) Water. Upon filling the vial with DI water, the measured raw transmission exceeded 100%. This is due to two main optical effects: the refractive index difference between the water and glass reduces surface reflection losses, and the curvature of the vial causes the fluid-filled cell to act as a lens, focusing light more effectively onto the detector.

Apparent transmission values above 100% are a known artifact in optical systems under certain conditions. This arises when inserting a curved, fluid-filled container between a well-aligned light source and detector improves light collection due to for example reduced Fresnel reflections and geometric focusing by curved surfaces. Regarding the reduced Fresnel reflections, when the space between the glass walls is filled with a fluid (e.g., water), the refractive index contrast at the glass-medium interfaces is reduced. This significantly lowers Fresnel reflection losses compared to air-glass interfaces, resulting in greater net light transmission through the vial. Regarding the geometric focusing by curved surfaces, when filled with fluid, the vial's curved surfaces behave like a simple positive lens. This lensing effect concentrates or collimates the light beam toward the detector's aperture. As a result, more light is directed into the detector than in the absence of the fluid, increasing the transmission. These two effects are multiplicative. The combination of near-lossless surface transmission and enhanced beam convergence can yield raw transmission values well above 100%, often ranging from 120% to over 200%.

Aspects of the present disclosure relate to a method for real-time detection and characterization of mineral scale formation using vertical turbidity scanning combined with transmission stability index (TSI) analysis. The turbidity scanner method as described herein provides continuous and real-time monitoring of turbidity variations and particulate behavior in fluid samples, providing insights into the onset and progression of scale formation, according to certain embodiments. Light transmission and scattering were analyzed to detect early-stage mineral precipitation, allowing high sensitivity to change in fluid clarity and particle distribution. A range of sea water-freshwater (SW-FW) ratios were evaluated, and the method successfully distinguished between conditions favorable for scale formation and those associated with fluid stability, according to certain embodiments. Transmission Stability Index values were used to quantify these observations, where lower TSI values corresponded to less scale formation and higher values indicated more mineral precipitation. The method enables real-time operational adjustments during testing to control scale accumulation, maintain flow assurance, and support stable operational efficiency.

Traditional approaches fail to detect early nucleation events or distinguish between suspended haze and settled scale, while techniques herein provide non-invasive and real-time detection capability. The turbidity-based data further support kinetic analysis of precipitation behavior, allowing improved water management strategies and enhanced operational performance in oil field environments. This is supported by experimental datasets, where specific SW-FW ratios exhibit turbidity profiles that correlate directly with stable conditions or the presence of scaling phenomena. The integration of high-resolution vertical turbidity scanning with spatially and temporally resolved TSI analysis addresses limitations in prior art that relied solely on bulk turbidity or single-point derivatives. The dual-domain monitoring capability enables localization of nucleation sites, differentiation between colloidal haze and crystalline precipitation, and detailed kinetic profiling. The disclosed method provides substantial advantages in field-scale deployment for flow assurance, particularly in upstream oil and gas applications, and represents a significant advancement in mineral scale management and diagnostic technologies.

Numerous modifications and variations of the present disclosure are possible in light of the above teachings. It is therefore to be understood that within the scope of the appended claims, the invention may be practiced otherwise than as specifically described herein.

1. A method for detecting and monitoring scale formation in a fluid mixture, comprising:
mixing a first solution and a second solution to form the fluid mixture, wherein the first solution has a first Ca²⁺ concentration that is 10% or less of a second Ca²⁺ concentration of the second solution, and a first SO₄²⁻ concentration that is 1000% or more of a second SO₄²⁻ concentration of the second solution;
scanning along a height direction of the fluid mixture to measure light transmission values of the fluid mixture as a function of time and height position; and
determining whether scale formation occurs in the fluid mixture based on the light transmission values.

2. The method of claim 1, further comprising:
calculating a transmission stability index (TSI) value based on a difference between consecutive transmission profiles of the light transmission values along the height direction of the fluid mixture.

3. The method of claim 2, wherein
$$SI ( {t}_{n} ) = {\sum }_{j = 1}^{n} \frac{{\sum }_{{z}_{min}}^{{z}_{max}} \vert I ( {t}_{j} , {z}_{i} ) - I ( {t}_{j - 1} , {z}_{i} ) \vert}{{z}_{n}} ,$$
where:
SI(tₙ) is the TSI value at time of tₙ,
I(tⱼ, zᵢ) is transmitted light intensity at time of tⱼ and position of zᵢ,
I(tⱼ₋₁, zᵢ) is transmitted light intensity at time of tⱼ₋₁ and position of zᵢ,
zₘₐₓ is a highest height position along the height direction of the fluid mixture,
zₘᵢₙ is a lowest height position along the height direction of the fluid mixture,
zₙ=zₘₐₓ−zₘᵢₙ and represents a height of the fluid mixture,
tₙ, tⱼ and tⱼ₋₁ respectively represent time during nth, jth and (j-1)th scanning cycles, and
n is an integer of 2 or more and represents a total number of scanning cycles.

4. The method of claim 3, further comprising:
determining that the scale formation occurs in the fluid mixture when the TSI value exceeds a predetermined threshold value over a defined time window.

5. The method of claim 3, further comprising:
determining that the scale formation occurs in the fluid mixture when the TSI value exceeds 1 over an initial five-hour time of the mixing.

6. The method of claim 1, further comprising:
calculating an average of the light transmission values at a bottom portion of the fluid mixture as a function of time, wherein the bottom portion has a bottom height along the height direction, which is 20% or less of a height of the fluid mixture along the height direction.

7. The method of claim 6, further comprising:
determining that the scale formation occurs in the fluid mixture when the average of the light transmission values at the bottom portion of the fluid mixture decreases by at least 10% during an initial five-hour time of the mixing.

8. The method of claim 6, further comprising:
determining that the scale formation occurs in the fluid mixture when the average of the light transmission values at the bottom portion of the fluid mixture decreases by at least 2% per hour.

9. The method of claim 1, wherein:
the light transmission values as a function of the vertical position have a peak value and a plateau value, and
the peak value is larger than the plateau value during an initial stage of the mixing.

10. The method of claim 9, further comprising:
monitoring the peak value and the plateau value during the mixing; and
determining that the scale formation occurs in the fluid mixture when the peak value first equals the plateau value.

11. The method of claim 1, wherein:
the first solution comprises:
$$5 - 35 g / L of {Na}^{+} ,$$
$$10 - 80 g / L of {Cl}^{-} ,$$
$$0.1 - 1.2 g / L of {Ca}^{2 +} ,$$
$$1 - 6 g / L of {Mg}^{2 +} ,$$
$$0.05 - 0.5 g / L of {HCO}_{{3}^{-}} , and$$
$$3 - 15 g / L of {SO}_{4}^{2 -} ;$$
and
the second solution comprises:
$$30 - 60 g / L of {Na}^{+} ,$$
$$100 - 300 g / L of {Cl}^{-} ,$$
$$15 - 25 g / L of {Ca}^{2 +} ,$$
$$1 - 8 g / L of {Mg}^{2 +} ,$$
$$0.05 - 0.5 g / L of {HCO}_{{3}^{-}} , and$$
$$0.01 - 0.5 g / L of {SO}_{4}^{2 -} .$$

12. The method of claim 11, wherein:
the first solution comprises:
$$18.29 g / L of {Na}^{+} ,$$
$$39.14 g / L of {Cl}^{-} ,$$
$$0.58 g / L of {Ca}^{2 +} ,$$
$$2.88 g / L of {Mg}^{2 +} ,$$
$$0.12 g / L of {HCO}_{{3}^{-}} , and$$
$$4.29 g / L of {SO}_{4}^{2 -} ;$$
and
the second solution comprises:
$$59.48 g / L of {Na}^{+} ,$$
$$151.02 g / L of {Cl}^{-} ,$$
$$17.02 g / L of {Ca}^{2 +} ,$$
$$3.33 g / L of {Mg}^{2 +} ,$$
$$0.35 g / L of {HCO}_{{3}^{-}} , and$$
$$0.35 g / L of {SO}_{4}^{2 -} .$$

13. The method of claim 1, wherein:
the first solution has a first total dissolved solid (TDS) concentration that is 50% or less of a second TDS concentration of the second solution,
the first TDS concentration is 30,000-130,000 ppm, and
the second TDS concentration is 150,000-400,000 ppm.

14. The method of claim 13, wherein:
the first TDS concentration is 65,000 ppm, and
the second TDS concentration is 233,000 ppm.

15. The method of claim 1, wherein:
the scale formation occurs at least as a result of precipitation of CaSO₄.

16. The method of claim 1, wherein:
the scanning is executed for a scanning duration of at least 5 hours at a scanning interval of at most 10 minutes.

17. The method of claim 16, wherein:
the scanning duration is 12 hours to 48 hours, and
the scanning interval is 5 seconds to 1 minute.

18. The method of claim 1, wherein the first solution is sea water, and the second solution is formation water present in an underground formation, the method further comprising:
recovering hydrocarbons from the underground formation, which comprises injecting the sea water into the underground formation so that the sea water and the formation water present in the underground formation mix with each other to form the fluid mixture.

19. The method of claim 18, further comprising:
determining in real time whether the scale formation occurs in the fluid mixture based on the light transmission values during the recovering.

20. The method of claim 18, further comprising:
installing a light source and a light detector adjacent to a production well for the underground formation; and
moving the light source and the light detector along a bottom portion of the production well in a height direction of the production well during the scanning.
